(19) United States
(12) Patent Application Publication
(10) Pub. No.: US 2022/0231069 A1
(43) Pub. Date: Jul. 21, 2022

(54) IMAGE-SENSING DEVICE

(71) Applicant: Silicon Optronics, Inc., Hsinchu (TW)

(72) Inventor: Bo-Ray LEE, Hsinchu (TW)

(21) Appl. No.: 17/581,139

(22) Filed: Jan. 21, 2022

(30) Foreign Application Priority Data
Jan. 21, 2021 (TW) 110102272

(51) Int. Cl. H01L 27/146; G01N 33/50

(52) U.S. Cl. CPC H01L 27/14636; H01L 27/1462; G01N 33/50

(57) ABSTRACT

Image-sensing devices are provided. An image-sensing device includes a substrate, a first dielectric layer, an image sensor array, a plurality of nanowells and a plurality of electrodes. The first dielectric layer is formed on the substrate, and has a first side and a second side. The image sensor array is formed between the substrate and the second side of the first dielectric layer, and includes a plurality of image-sensing cells. The nanowells are formed in the first dielectric layer, and each of the nanowells has an opening on the first side of the first dielectric layer. Each of the electrodes extends from the second side to the first side of the first dielectric layer and is located between two adjacent nanowells.

### CROSS REFERENCE TO RELATED APPLICATIONS

This Application claims priority of Taiwan Patent Application No. 110102272, filed on Jan. 21, 2021. the entirety of which is incorporated by reference herein.

### BACKGROUND OF THE INVENTION

### Field of the Invention

The invention relates to an image-sensing device, and more particularly to an image-sensing device with nanowells.

### Description of the Related Art

An image sensor is a semiconductor device that converts light images into electrical signals. Image sensors can generally be classified as either charge-coupled devices (CCD) or complementary metal-oxide-semiconductor (CMOS) image sensors, Among these image sensors, complementary metal-oxide-semiconductor image sensor includes a photodiode for detecting incident light and converting it into an electrical signal, and a logic circuit for transmitting and processing the electrical signal.

In addition to the general purpose of simply sensing images, more and more image sensors have been applied to various inspection tasks, such as biomedical inspections. Specifically, various characteristics of the object to be tested can be detected or determined by the light excited by the object to be tested after being irradiated by an external light source.

However, when the size of the sensing cell or pixel of the image sensor is reduced, there will be, for example, cross-talk, photon response non-uniformity (PRNU), low signal-to-noise ratio (SNR), and other issues. Therefore, an image-sensing device that can improve performance is desired.

### BRIEF SUMMARY OF THE INVENTION

Image-sensing devices are provided. An embodiment of an image-sensing device is provided. The image-sensing device includes a substrate, a first dielectric layer, an image sensor array, a plurality of nanowells and a plurality of electrodes. The first dielectric layer is formed on the substrate, and has a first side and a second side opposite to the first side. The image sensor array is formed between the substrate and the second side of the first dielectric layer, and includes a plurality of image-sensing cells. The nanowells are formed in the first dielectric layer, and each of the nanowells has an opening on the first side of the first dielectric layer. Each of the electrodes extends from the second side to the first side of the first dielectric layer and is located between two adjacent nanowells.

Moreover, an embodiment of an image-sensing device is provided. The image-sensing device includes a substrate, an image sensor array, a first dielectric layer, a first passivation layer, a second dielectric layer, a plurality of nanowells and a plurality of electrodes. The image sensor array is -formed on the substrate, and comprising a plurality of image-sensing cells. The first dielectric layer is formed on the image sensor array. The first passivation layer is formed on the first dielectric layer. The second dielectric layer is formed on the first passivation layer. The nanowells are formed in the second dielectric layer, and each of the nanowells has an opening on the upper surface of the second dielectric layer. Each of the electrodes extends from the first dielectric layer through the first passivation layer to the second dielectric layer and is disposed between two adjacent nanowells.

A detailed description is given in the following embodiments with reference to the accompanying drawings.

### BRIEF DESCRIPTION OF DRAWINGS

The invention can be more fully understood by reading the subsequent detailed description and examples with references made to the accompanying drawings, wherein:

FIG. 1 shows a cross-sectional view of an image-sensing device according to some embodiments of the invention.

FIG. 2A shows a rolling shutter image-sensing cell according to some embodiments of the invention.

FIG. 2B shows a global shutter image-sensing cell according to some embodiments of the invention.

FIG. 3 shows a top view of the dipoles of the object to be tested (e.g., the object to be tested of FIG. 1) in the nanowell array when the electrode of the image-sensing device in FIG. 1 is operated in an unbiased mode according to some embodiments of the invention.

FIG. 4 shows a top view of dipoles of the object to be tested (e.g., the object to be tested in FIG. 1) in the nanowell array when the electrodes of the image-sensing device in FIG. 1 are operated in a first bias mode according to some embodiments of the invention.

FIG. 5 shows a top view of dipoles of the object to be tested (e.g., the object to be tested in FIG. 1) in the nanowell array when the electrodes of the image-sensing device in FIG. 1 are operated in a second bias mode according to some embodiments of the invention.

FIGS. 6A through 6D show the top views of dynamically assigning electrodes around the nanowells in a third bias mode according to some embodiments of the invention.

FIGS. 7A through 7D show the top views of dynamically assigning electrodes around the nanowell in a fourth bias mode according to some embodiments of the invention.

FIG. 8 shows a cross-sectional view of the nano-well and the surrounding electrodes according to some embodiments of the invention.

FIGS. 9A through 9F show cross-sectional views illustrating a semiconductor structure forming the image-sensing device according to some embodiments of the invention.

FIG. 10 shows a cross-sectional view of a semiconductor structure forming the image-sensing device according to some embodiments of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

It should be understood that, the elements or devices of the drawings may exist in various forms well known to those skilled in the art. In addition, relative terms such as “lower” or “bottom” and “higher” or “top” may be used in the embodiments to describe the relative relationship between one element of the figure and another element. It can be understood that if the illustrated device is turned upside down and turned upside down, the element described on the “lower” side will become the element on the “higher” side. The embodiments of the disclosure can be understood together with the drawings, and the drawings of the disclosure are also considered as a part of the disclosure description. It should be understood that the drawings disclosed in this disclosure are not drawn to scale. In fact, the dimensions of the elements may be arbitrarily enlarged or reduced in order to clearly show the features of the present invention.

Furthermore, the elements or devices of the drawings may exist in various forms well known to those skilled in the art. Moreover, understandably, although the terms “first”, “second”, “third”, etc. may be used herein to describe various elements or parts, these elements, components, or parts should not be limited by these terms, and these terms are only Is used to distinguish different elements, components, areas, layers or parts. Therefore, a first element, component, area, layer or part discussed below may be referred to as a second element, component, area, layer or part without departing from the teachings of this disclosure.

In some embodiments of the present disclosure, terms such as “connect” and “interconnect” with regard to bonding and connection may refer to the two structures being in direct contact, or may refer to the two structures not being in direct contact unless specifically defined. There are other structures between these two structures. In addition, the term “joining and connecting” may also include a case where both structures are movable or both structures are fixed.

It should he understood that when an element or layer is referred to as being “on” or “connected” with another element or layer, it can be directly on or directly connected to another element or layer. The layers are connected, or there may also be intervening elements or layers. Conversely, when an element is referred to as being “directly” on or on another element or “directly” connected to another element or layer, there are no intervening elements.

Unless otherwise defined, all terms (including technical and scientific terms; used herein have the same meaning as commonly understood by those skilled in the art to which this disclosure belongs. It is understandable that these terms, such as those defined in commonly used dictionaries, should be interpreted as having a meaning consistent with the background or context of the related technology and this disclosure. It should not be interpreted in an idealized or excessively formal manner unless specifically defined in the disclosed embodiments.

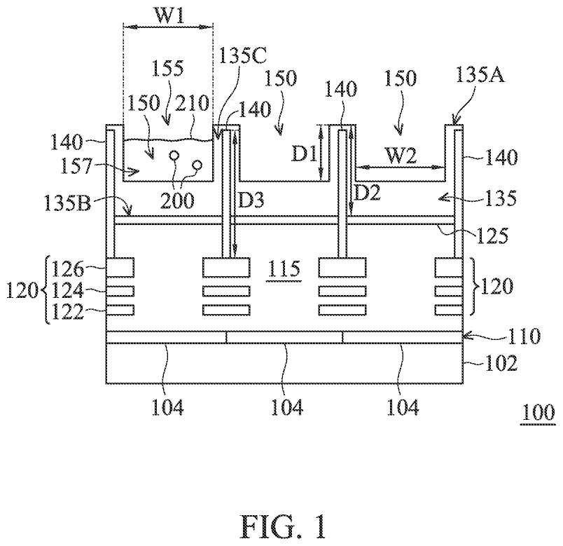

FIG. 1 shows a cross-sectional view of an image-sensing device 100 according to some embodiments of the invention. It should be understood that, according to some embodiments, additional features may be added to the image-sensing device 100 described below. According to some embodiments, some features described below may be replaced or deleted.

As shown in FIG. 1, the image-sensing device 100 includes a substrate 102. In some embodiments, the substrate 102 is a semiconductor substrate. For example, the material of the substrate 102 may include monocrystalline, polycrystalline, or amorphous silicon (Si) or germanium (Ge) or a combination thereof. In some embodiments; the substrate 102 is formed of a compound semiconductor. For example, in some embodiments, the material of the substrate 100 may include silicon carbide (SiC), gallium arsenide (GaAs), gallium phosphide (GaP), indium phosphide (InP), indium arsenide (InAs), or a combination thereof. In addition, according to some embodiments, the material of the substrate 102 may be formed of alloy semiconductors. For example, in some embodiments, the material of the substrate 102 may include germanium silicide (SiGe), aluminum gallium arsenide (AlGaAs), gallium indium arsenide (GaInAs), gallium indium phosphide (GaInP), gallium arsenide (GaAsP)) or a combination thereof.

In FIG. 1, the image-sensing device 100 further includes an image sensor array 110 formed on the substrate 102. In some embodiments, some components (or elements) of the image sensor array 110 is disposed in the substrate 102. The image sensor array 110 includes a plurality of image-sensing cells 104 arranged in multiple rows and multiple columns, and each image-sensing cell 104 includes a photodiode. The photodiode is configured to receive light and convert it into electrical signals. In some embodiments, the image-sensing cell 104 may be a rolling shutter image-sensing cell or a global shutter image-sensing cell.

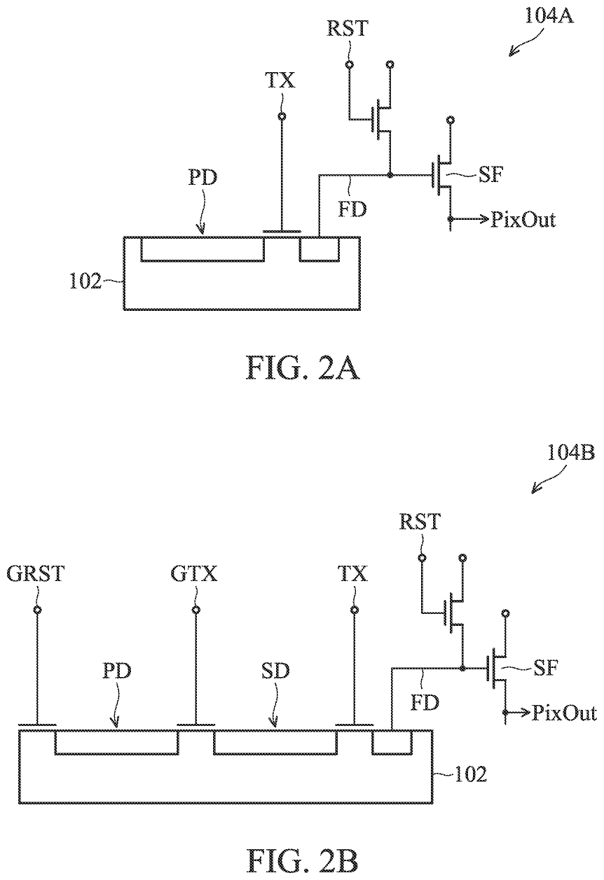

Referring to FIGS. 2A and 2B, FIG. 2A shows a rolling shutter image-sensing cell 104A according to some embodiments of the invention, and FIG. 2B shows a global shutter image-sensing cell 104B according to some embodiments of the invention. In the image-sensing cells 104A and 104B, the photodiode PD may include the source and drain of a metal oxide semiconductor (MOS) transistor, and the source and drain of the MOS transistor are configured to transmit current to other components, such as other MOS transistors. In some embodiments, the image-sensing cells 104A and 104B may include a transmission gate TX, a reset gate RST, a floating diffusion FD, a source follower SF, or a combination thereof. Furthermore, the image-sensing cells 104A and 104B are further coupled to external devices or circuits, so as to transmit the output signal PixOut to other circuits, such as a signal processor (not shown). It is noted that FIGS. 2A and 2B only simply show some components of the image-sensing cells 104A and 104B, and are not intended to limit the invention. Any image-sensing cell suitable for rolling shutters or global shutters can be used as the image-sensing cell of the invention.

Referring back to FIG. 1, the image-sensing device 10 further includes a dielectric layer 115, and the dielectric layer 115 is formed on the image sensor array 110. In other words, the dielectric layer 115 is configured to cover the image-sensing cells 104 of the image sensor array 110. In some embodiments, the material of the dielectric layer 115 may include silicon oxide, silicon nitride, silicon oxynitride, high-k dielectric materials, other suitable dielectric materials, or the foregoing combination. In some embodiments, the high dielectric constant dielectric material may include metal oxide, metal nitride, metal silicide, metal aluminate, zirconium silicate, zirconium aluminate, or a combination thereof.

In some embodiments, the dielectric layer 115 is formed by the physical vapor deposition (PVD), chemical vapor deposition (CVD), coating process, other suitable method, or a combination thereof. The physical vapor deposition process may include, for example, a sputtering process, an evaporation process, or pulsed laser deposition. The chemical vapor deposition process may include, for example, a low pressure chemical vapor deposition process (LPCVD), a low temperature chemical vapor deposition process (LLCM), a rapid temperature rise chemical vapor deposition process (RTCVD), a plasma assisted chemical vapor deposition process (PECVD), or atomic layer deposition process (ALD) and so on.

In FIG. 1, the image-sensing device 100 further includes an interconnect structure 120, and the interconnect structure 120 is formed in the dielectric layer 115. In some embodiments, the projection (not shown) of the interconnect structure 120 on the substrate 102 is overlapped between two adjacent image-sensing cells 104, that is, the edge of the image-sensing cells 104. In some embodiments, the interconnect structure 120 includes a plurality of conductive layers 122, 124, and 126. Each of the conductive layers 122, 124, and 126 includes a plurality of conductive electrodes, so as to transmit signals in the image-sensing cells 104 of the image-sensing device 100 and related circuits. In FIG. 1, the conductive layer 122 is the lowest conductive layer adjacent to the image sensor array 110, and the conductive layer 126 is the highest conductive layer away from the image sensor array 110. In addition, the conductive layer 124 is an intermediate conductive layer disposed between the conductive layers 122 and 126. It should be understood that although three layers of conductive layers 122, 124, and 126 are exemplarily illustrated, the invention is not limited thereto. In accordance with different embodiments, according to need, suitable amount and structure of the conductive layer may be arranged to form the interconnect structure 120.

In some embodiments, the interconnect structure 120 may include a metallic conductive material, a transparent conductive material, or a combination thereof. The metallic conductive material may include copper (Cu), aluminum (Al), gold (Au), silver (Au), titanium (Ti), tungsten (W), molybdenum (Mo), nickel (Ni), copper alloy, aluminum alloy, gold alloy, silver alloy, titanium alloy, tungsten alloy, molybdenum alloy, nickel alloy, or a combination thereof. The transparent conductive material may include a transparent conductive oxide (TCO). For example, the transparent conductive oxide may include indium tin oxide (ITT), tin oxide (SnO), zinc oxide (ZnO), indium zinc oxide (IZO), indium gallium zinc oxide (IGZO), indium tin zinc oxide (ITZO), antimony tin oxide (ATO), antimony zinc oxide (AZO), or a combination thereof.

In some embodiments, a physical vapor deposition (PVD) process, a chemical vapor deposition (CVD) process, a coating process, other suitable processes, or a combination thereof may be used to form the interconnect structure 120. In some embodiments, a patterning process may be used to form the interconnect structure 120. In some embodiments, the patterning process may include a photolithography process and an etching process. The photolithography process may include, but is not limited to, photoresist coating (for example, spin coating), soft baking, hard baking, mask alignment, exposure, post-exposure baking, photoresist development, cleaning, and drying. The etching process may include a. dry etching process or a wet etching process, but it is not limited thereto.

In FIG. 1, the image-sensing device 100 further includes a passivation layer 125, and the passivation layer 125 is formed over the dielectric layer 115. In some embodiments, the passivation. layer 125 may include silicon nitride (Si₃N₄), silicon oxide (SiO₂), silicon oxynitride (SiON), aluminum oxide (Al₂O₃), aluminum nitride (AlN), polyimide (PI), benzocyclobutene (BCB), polybenzoxazole (PBO), other dielectric materials, or a combination thereof. In some embodiments, metal organic vapor deposition methods, chemical vapor deposition methods (such as low pressure chemical vapor deposition or plasma assisted chemical vapor deposition), spin coating methods, other appropriate methods, or combinations thereof can be used to form the passivation layer 125 on the dielectric layer 115. The passivation layer 125 can protect the underlying structure, serve as a buffer between the structure to be formed later, and provide physical isolation and structural support.

In FIG. 1, the image-sensing device 100 further includes a dielectric layer 135, and the dielectric layer 135 is formed on the passivation layer 125. The dielectric layer 135 has a first side 135A and a second side 135B, and the first side 135A is opposite to the second side 135B. In some embodiments, the first side 135A of the dielectric layer 135 is the upper surface, and the second side 135B of the dielectric layer 135 is the lower surface. The second side 135B of the dielectric layer 135 is in contact with the passivation layer 125. In some embodiments, the material of the dielectric layer 135 may include silicon oxide, silicon nitride, silicon oxynitride, high-k dielectric materials, other suitable dielectric materials, or combinations thereof. In some embodiments, the high-k dielectric material may include metal oxides, metal nitrides, metal silicides, metal aluminates, zirconium silicates, zirconium aluminates, or combinations thereof.

In FIG. 1, the image-sensing device 100 further includes a plurality of nanowells 150. Each nanowell 150 has an opening 155 on the first side 335A of the dielectric layer 135. Furthermore, there is a depth (or thickness) D1 between the bottom surface 157 of the nanowell 150 and the opening 155, and the dielectric layer 135 has a depth D2 greater than the depth D1, that is, D2>D1. In some embodiments, the width (or diameter) W1 of the opening 155 is equal to the width W2 of the bottom surface 157, that is, W1=W2. In some embodiments, the width W of the opening 155 is greater than the width W2 of the bottom surface 157, that is, W1>W2. Moreover, the nanowells 150 are separated by the first portion 135C of the dielectric layer 135.

When the object to be tested 200 is filled into the nanowells 150, it can be excited by the excitation light from the upper light source (not shown). After the object to be tested 200 is excited, the object to be tested 200 emits light in a specific wavelength range, and the emitted light can be detected by the image-sensing cells 104 to determine the characteristics of the object to be tested 200. In some embodiments, the object to be tested 200 may be included in the sample solution (or chemical liquid) 210 filled in the nanowells 150.

In different embodiments, according to the characteristics of the tag of the object to be tested 200, excitation light with a suitable wavelength or frequency range is provided. For example, the tag can be excited to generate fluorescence or luminescence, but the present invention is not limited thereto. In some embodiments, the light source (not shown) may include polarized light, unpolarized light, or a combination thereof.

In some embodiments, the object to be tested 200 may include a biological molecule, a chemical molecule, or a combination thereof. For example, in some embodiments, the object to be tested 200 may include deoxyribonucleic acid (DNA), ribonucleic acid (RNA), proteins, cells, other organic and inorganic small molecules, or a combination thereof, but the present disclosure is not limited thereto. Moreover, in some embodiments, the object to be tested 200 may include a fluorescent marker.

In FIG. 1, the image-sensing device 100 further includes a plurality of electrodes 140. In some embodiments, the electrode 140 may be a via or a contact. In addition, each electrode 140 is in contact with and formed on the conductive layer 126 of the interconnect structure 120, and extends to the first side 135A of the dielectric layer 135 through the dielectric layer 115, the passivation layer 125, and the dielectric layer 135 in sequence until reaching the first part 135C of the dielectric layer 135. In other words, each electrode 140 is disposed between two adjacent nanowells 150. In addition, the depth D3 of the electrode 140 is greater than the depth D2 of the dielectric layer 135, that is, D3>D2.

In the image-sensing device 100, the image-sensing cell 104 can detect the light emitted by the object to be tested 200. By controlling the voltage (or bias and polarity) of the electrode 140, an electric field is generated in the nanowell 150 to control the direction of the dipoles of the object to be tested 200, so as to reduce the influence of cross-talk. Furthermore, by periodically adjusting the voltage of the electrode 140, the dipole moment and/or the moment of inertia of the object to be tested 200 are obtained. Therefore, in addition to the light emitted by the object to be tested 200, the image-sensing device 100 can also determine the characteristics of the object to be tested 200 according to the dipole moment and/or the moment of inertia of the object to he tested 200, so as to identify the object to be tested 200.

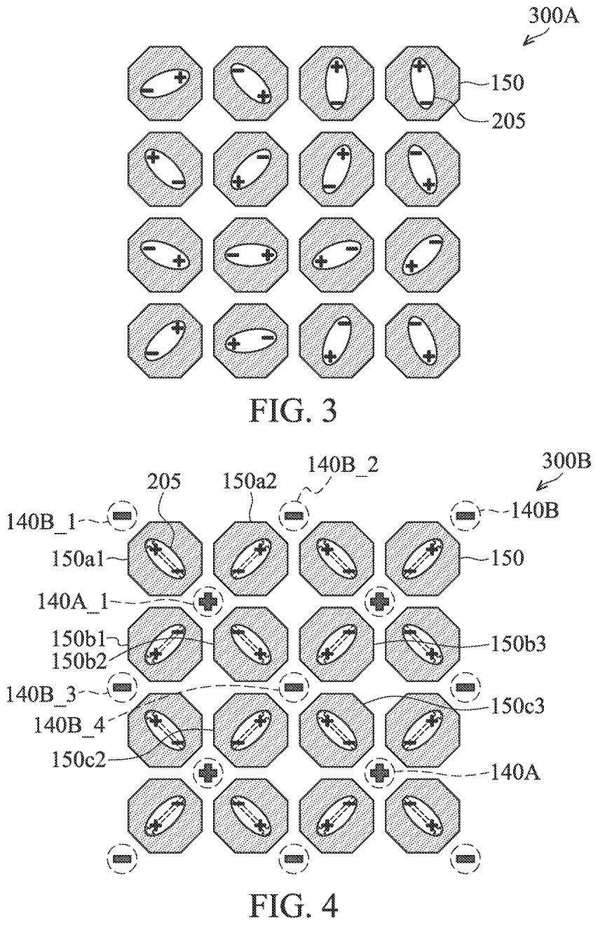

FIG. 3 shows a top view of the dipoles 205 of the object to be tested (e.g., the object to be tested 200 of FIG. 1) in the nanowell array 300A when the electrode 140 of the image-sensing device 100 in FIG. 1 is operated in an unbiased mode according to some embodiments of the invention. In FIG. 3, since no bias is applied to the electrodes 140, the electrodes 140 are not shown. In addition, it should be noted that the nanowell array 300A in FIG. 3 shows a 4×4 array of nanowells 150. In other embodiments, the nanowell array 300A may include a greater or lesser number of nanowells 150. The dipole 205 is a structure formed by two positively and negatively charged particles with a short distance. As shown in FIG. 3, when the electrodes 140 are operated in the unbiased mode, the dipole 205 in each nanowell 150 is randomly arranged. Therefore, the sum of the dipoles 205 in the nanowell array 300A is randomly polarized, which is prone to crosstalk and causes high photon response non-uniformity (PRNU). Moreover, when the electrodes 140 are operated in the unbiased mode, the direction of the dipole 205 in the nanowell 150 is also unpredictable.

In the image-sensing device 100, the shape of the nanowell 150 is a regular octagon. In some embodiments, the shape of the nanowell 150 is an equilateral polygon. In some embodiments, the shape of the nanowell 150 is an equilateral polygon with more than three sides. In some embodiments, the shape of the nanowell 150 is circular.

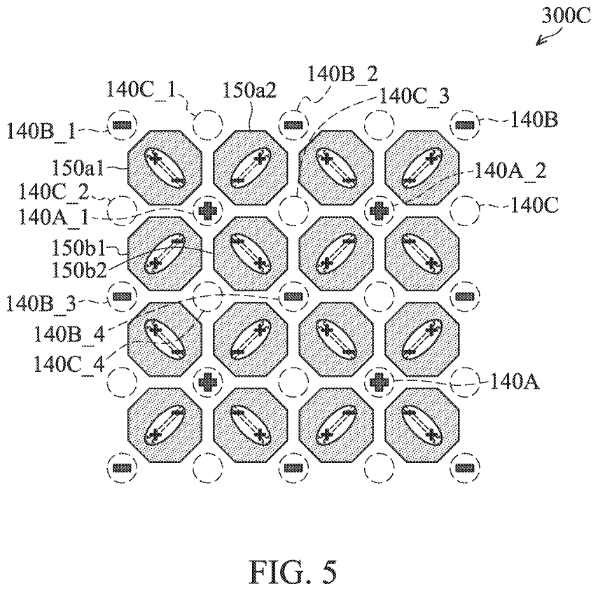

FIG. 4 shows a top view of dipoles 205 of the object to be tested (e.g., the object to be tested 200 in FIG. 1) in the nanowell array 300B when the electrodes 140 of the image-sensing device 100 in FIG. 1 are operated in a first bias mode according to some embodiments of the invention. It is worth noting that the nanowell array 300B in FIG. 4 shows the nanowell 150 of the 4×4 array. in other embodiments, the nanowell array 300B may include a greater or lesser number of nanowells 150. As shown in FIG. 4, the electrode 140A represents the electrode 140 with a high voltage, and the electrode 140B represents the electrode 140 with a low-voltage. In some embodiments, the electrode 140A has a positive voltage (e.g., +3V), and the electrode 140B has a negative voltage (e.g., −3V). In some embodiments, the electrode 140A has a voltage (e.g., 5V) greater than the ground voltage, and the electrode 140B has a ground voltage (e.g., 0V). In some embodiments, the voltages of the electrode 140A and the electrode 140B can be changed or interchanged over time. For example, at a first time point, the electrode 140A has a positive voltage and the electrode 140B has a negative voltage. Next, at the second time point, the electrode 140A has a negative voltage and the electrode 140B has a positive voltage.

In the nanowell array 300B, each nanowell 150 is surrounded by an electrode 140A and an electrode 140B, and the voltage of the electrode 140A is greater than the voltage of the electrode 140B. Therefore, in each nanowell 150, when the applied electric field (as indicated by the arrow) is large enough, the direction of the dipole 205 of the object to be tested (such as the object to be tested 200 in FIG. 1) is from the electrode 140A with a high voltage to the electrode 140B with a low voltage. For example, for the nanowell 150a1, the nanowell 150a1 is surrounded by the electrode 140A_1 and the electrode 140B_1, and the electrode 140A_1 is arranged at the lower right of the nanowell 150a1 and the electrode 140B_1 is arranged at the upper left of the nanowell 150a1. Therefore, the direction of the dipole 205 in the nanowell 150a1 is from the electrode 140A_1 to the electrode 140B_1 (i.e., from lower right to upper left). Similarly, the nanowell 150a2 is surrounded by the electrodes 140A_1 and 140B_2, and the electrode 140A_1 is arranged at the lower left of the nanowell 150a2 and the electrode 140B_2 is arranged at the upper right of the nanowell 150a2. Therefore, the direction of the dipole 205 in the nanowell 150a2 is from the electrode 140A_1 to the electrode 140B_2 (i.e., from lower left to upper right). In addition, the nanowell 150b1 is surrounded by the electrodes 140A_1 and 140B_3, and the electrode 140A_1 is arranged at the upper right of the nanowell 150b1 and the electrode 140B_3 is arranged at the lower left of the nanowell 150b1. Therefore, the direction of the dipole 205 in the nanowell 150b1 is from the electrode 140A_1 to the electrode 140B_3 (i.e. from the upper right to the lower left). Furthermore, the nanowell 150b2 is surrounded by the electrodes 140A_1 and 140B_4, and the electrode 140A_1 is arranged at the upper left of the nanowell 150b2 and the electrode 140B_4 is arranged al the lower right of the nanowell 150b2. Therefore, the direction of the dipole 205 in the nanowell 150b2 is from the electrode 140A_1 to the electrode 140B_4 (i.e. from the upper left to the lower right).

In the nanowell array 300B each electrode 140 disposed inside the array is surrounded by four nanowells. For example, the electrode 140A_1 is surrounded by four nanowells 150a1, 150a2, 150b1, and 150b2 that is, the electrode 140A_1 is arranged between the nanowells 150a1, 150a2, 150b1 and 150b2. Similarly, the electrode 140B_4 is surrounded by four nanowells 150b2, 150b3, 150c2, and 150c3, that is, the electrode 140B_4 is arranged between the nanowells 150b2, 150b3, 150c2, and 150c3.

In the first bias mode, the electrodes 140B are arranged (or assigned) in odd rows of the electrode array, and the electrodes 140A are arranged (or assigned) in even rows of the electrode array. For example, the electrodes 140B_1 and 140B_2 are arranged in the first row of the electrode array, and the electrode 140A_1 is arranged in the second row of the electrode array. In addition, the electrodes 140B are arranged (or assigned) in odd columns of the electrode array, and the electrodes 140A are arranged (or assigned) in even columns of the electrode array. For example, the electrodes 140B_1 and 140B_2 are respectively arranged in the first column and the third column of the electrode array, and the electrode 140A_1 is arranged in the second column of the electrode array. In other words, the electrodes 140A and 140B are assigned on staggered lines (e.g., staggered columns and rows). By assigning the electrodes 140A and 140B and controlling the voltages of the electrodes 140A and 140B, the sum of the dipoles 205 in the nano-well array 300B is controllable, so the optical response signal distribution is controllable, and the crosstalk phenomenon can also be reduced.

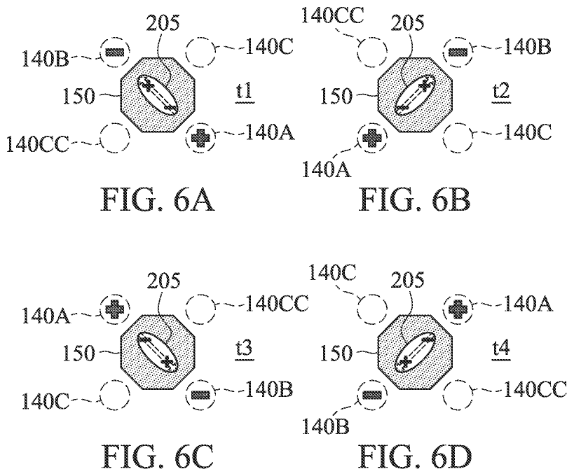

FIG. 5 shows a top view of dipoles 205 of the object to he tested (e.g., the object to be tested 200 in FIG. 1) in the nanowell array 300C when the electrodes 140 of the image-sensing device 100 in FIG. 1 are operated in a second bias mode according to some embodiments of the invention. It should be noted that the nanowell array 300C in FIG. 5 shows the nanowell 150 of the 4×4 array. In other embodiments, the nanowell array 300C may include a greater or lesser number of nanowells 150. The nanowell array 300C in FIG. 5 and the nanowell array 300B in FIG. 4 have similar configurations of electrodes 140A and 140B. The difference between the nanowell array 300C in FIG. 5 and the nanowell array 300B in FIG. 4 is that the nanowell array 300C further includes the electrodes 140C. In FIG. 5, the electrode 140A represents the electrode 140 having a high voltage, the electrode 140B represents the electrode 140 having a low voltage, and the electrode 140C represents the electrode 140 having an average voltage (or intermediate voltage). In some embodiments, the electrode 140A has a positive voltage (e.g., +3V), the electrode 140B has a negative voltage (e.g., −3V), and the electrode 140C has a ground voltage (e.g., 0V). In some embodiments, the electrode 140A has a higher voltage (e.g., 5V) the electrode 140B has a ground voltage (e.g., 0V), and the electrode 140C has an intermediate voltage (e.g., 2.5V, 3V, etc.).

In the nanowell array 300C, each nanowell 150 is surrounded by one electrode 140A, one electrode 140B, and two electrodes 140C. In addition, the voltage of the electrode 140A is greater than the voltage of the electrode 140C, and the voltage of the electrode 140C is greater than the voltage of the electrode 140B. Therefore, in each nanowell 150, the direction of the dipoles 205 of the object to be tested (not shown) is from the electrode 140A with a high voltage to the electrode 140B with a low voltage.

In FIG. 5, the nanowell 150a1 is surrounded by the electrode 140A_1, the electrode 140B_1 and the electrodes 140C_1 and 140C_2. The electrode 140A_1 is arranged on the lower right of the nanowell 150a1, the electrode 140C_1 is arranged on the upper right of the nanowell 150a1, the electrode 140B_1 is arranged on the upper left of the nanowell 150a1, and the electrode 140C_2. is arranged on the lower left of the nanowell 150a1. When the applied electric field (as indicated by the arrow) is large enough, the direction of the dipoles 205 of the object to be tested (such as the object to be tested 200 in FIG. 1) is from the electrode with a high voltage to the electrode with a low voltage. Therefore, the direction of the dipole 205 in the nanowell 150a1 is from the electrode 140A_1 to the electrode 140B_1 from lower right to upper left). Similarly, the nanowell 150a2 is surrounded by the electrode 140A_1. the electrode 140B_2, and the electrodes 140C_1 and 140C_3. The electrode 140A_1 is arranged on the lower left of the nanowell 150a2, the electrode 140C_1 is arranged on the upper left of the nanowell 150a2, the electrode 140B_2 is arranged on the upper right of the nanowell 150a2, and the electrode 140C_3 is arranged on the lower right of the nanowell 150a2. Therefore, the direction of the dipole 205 in the nanowell 150a2 is from the electrode 140A_1 to the electrode 140B_2 (i.e., from lower left to upper right).

In the second bias mode, the electrode 140B is arranged (or assigned) in the odd rows of the electrode array, and the electrode 140A is arranged (or assigned) in the even rows of the electrode array. In addition, the electrode 140B is arranged (or assigned) in the odd columns of the electrode array, and the electrode 140A is arranged (or assigned) in the even columns of the electrode array. Furthermore, the electrodes 140C are arranged (or assigned) in each row and each column of the electrode array. In the odd rows and the odd columns, the electrodes 140B and 140C are arranged alternately. In the even rows and the even columns, the electrodes 140A and 140C are arranged alternately. By using the electrode 140C, the direction of the dipole 205 in each nanowell 150 of the nanowell array 300C can be more fixed. Moreover, by assigning electrodes 140A, 140B and 140C and controlling the voltages of electrodes 140A, 140B and 140C, the sum of dipoles 205 in the nanowell array 300C is controllable, so the optical response signal distribution is controllable and the crosstalk phenomenon is also decreased.

In some embodiments, the voltage controller (not shown) of the image-sensing device 100 can fixedly assign the electrodes 140 disposed around each nanowell 150 as the electrodes 140A, 140B, or 140C, so that the direction of dipole 205 in the nanowell 150 will not change, In some embodiments, the voltage controller (not shown) of the image-sensing device 100 may dynamically assign the electrodes 140 disposed around each nanowell 150 as the electrodes 140A, 140B, or 140C, so as to change the direction of dipole 205 in the nanowell 150.

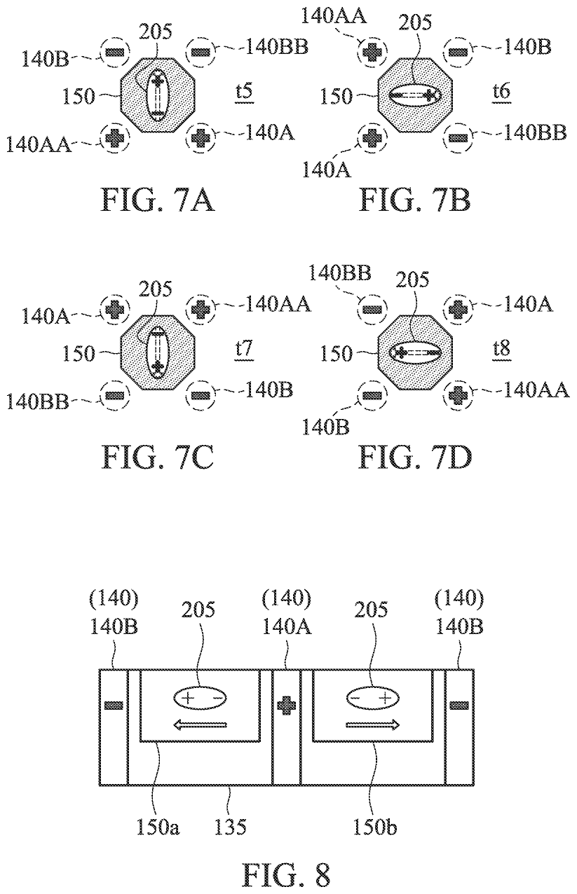

FIGS. 6A through 6D show the top views of dynamically assigning electrodes 140 around the nanowells 150 in a third bias mode according to some embodiments of the invention. By changing the voltages of at least four electrodes 140, the direction of the dipole 205 of the object to be tested (such as the object to be tested 200 in FIG. 1) is controlled in the nanowell 150.

FIG. 6A shows a schematic diagram illustrating the electrode 140 assigned at the first time t1. In FIG. 6A, the electrode 140 at the lower right of the nanowell 150 is assigned as the electrode 140A with a high voltage, and the electrode 140 at the lower left of the nanowell 150 is assigned as the electrode 140CC with an intermediate voltage. Furthermore, the electrode 140 on the upper left of the nanowell 150 is assigned as the electrode 140B with a low voltage, and the electrode 140 on the upper right of the nanowell 150 is assigned as the electrode 140C with an intermediate voltage. Therefore, the direction of the dipole 205 in the nanowell 150 is from the electrode 140A_1 at the lower right to the electrode 140B_1 at the upper left.

FIG. 6B shows a schematic diagram illustrating the electrode 140 assigned at the second time 12. In FIG. 6B, the electrode 140 at the lower left of the nanowell 150 is assigned as the electrode 140A with a high voltage, and the electrode 140 at the upper left of the nanowell 150 is assigned as the electrode 140CC with an intermediate voltage. Furthermore, the electrode 140 on the upper right of the nanowell 150 is assigned as the electrode 140B with a low voltage, and the electrode 140 on the lower right of the nanowell 150 is assigned as the electrode 140C with an intermediate voltage. Therefore, the direction of the dipole 205 in the nanowell 150 is from the electrode 140A_1 at the lower left to the electrode 140B_1 at the upper right. In other words, compared to FIG. 6A, the direction of dipole 205 is rotated 90 degrees clockwise.

FIG. 6C shows a schematic diagram illustrating the electrode 140 assigned at the third time t3, in FIG. 6C, the electrode 140 on the upper left of the nanowell 150 is assigned as the electrode 140A with a high voltage, and the electrode 140 on the upper right of the nanowell 150 is assigned as the electrode 140CC with an intermediate voltage. Furthermore, the electrode 140 at the lower right of the nanowell 150 is assigned as an electrode 140B with a low voltage, and the electrode 140 at the lower left of the nanowell 150 is assigned as an electrode 140C with an intermediate voltage. Therefore, the direction of the dipole 205 in the nanowell 150 is from the electrode 140A_1 at the upper left to the electrode 140B_1 at the lower right. In other words, compared to FIG. 6B, the direction of dipole 205 is rotated 90 degrees clockwise.

FIG. 6D shows a schematic diagram illustrating the electrode 140 assigned at the fourth time t4. In FIG. 6D, the electrode 140 at the upper right of the nanowell 150 is assigned as the electrode 140A with a high voltage, and the electrode 140 at the upper left of the nanowell 150 is assigned as the electrode 140C with an intermediate voltage. Furthermore, the electrode 140 at the lower left of the nanowell 150 is assigned as the electrode 140B with a low voltage, and the electrode 140 at the lower right of the nanowell 150 is assigned as the electrode 140CC with an intermediate voltage. Therefore, the direction of the dipole 205 in the nanowell 150 is from the electrode 140A_1 at the upper right to the electrode 140B_1 at the lower left. In other words, compared to FIG. 6C, the direction of dipole 205 is rotated 90 degrees clockwise.

Referring to FIGS. 6A through 6D together, by periodically assigning the fur electrodes 140 to the corresponding voltages at the first time t1, the second time t2, the third time t3, and the fourth time t4 in sequence, the dipole 205 in the nanowell 150 is rotated, for example, in a clockwise direction. It should be noted that a first time difference Δt1 from the first time t1 to the second time t2 is the same as a second time difference Δt2 from the second time t2 to the third time t3, and the second time difference Δt2 is the same as a third time difference Δt3 from the third time t3 to the fourth time t4. Furthermore, when the dipole 205 rotates, the image-sensing cell 104 can detect the change of the object to be tested, and then obtain the dipole moment and the moment of inertia of the object to be tested. Therefore, the image-sensing device 100 can identify the object to be tested more quickly according to the characteristics of the object to be tested.

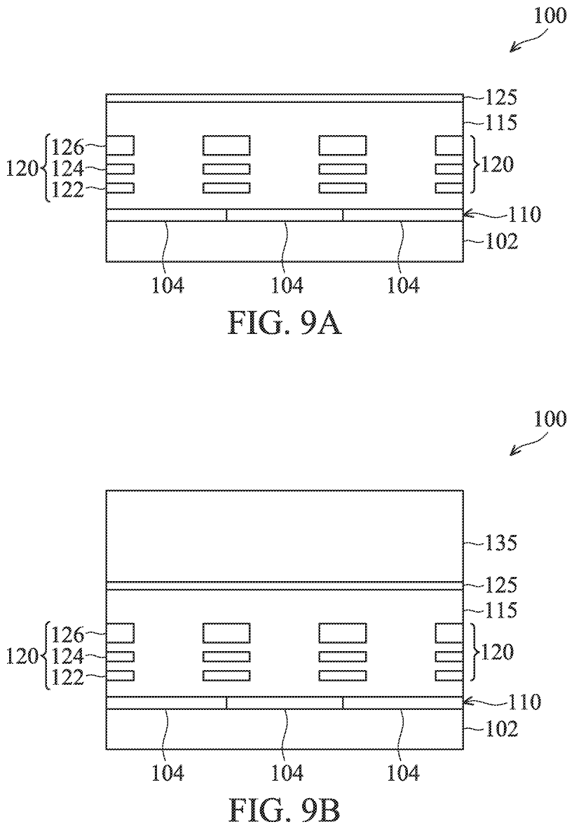

FIGS. 7A through 7D show the top views of dynamically assigning electrodes 140 around the nanowell 150 in a fourth bias mode according to some embodiments of the invention. By changing the voltages of at least four electrodes 140, the direction of the dipole 205 of the object to be tested (such as the object to be tested 200 in FIG. 1) is controlled in the nanowell 150.

FIG. 7A shows a schematic diagram illustrating the electrode 140 assigned at the fifth time t5. In FIG. 7A, the electrodes 140 at the lower right and lower left of the nanowell 150 are assigned as the electrodes 140A and 140AA with a high voltage, respectively. Furthermore, the electrodes 140 on the upper left and upper right of the nanowell 150 are respectively assigned as the electrodes 140B and 140BB with a low voltage. Therefore, the direction of the dipole 205 in the nanowell 150 is from below to upward.

FIG. 7B shows a schematic diagram illustrating the electrode 140 assigned at the sixth time t6. In FIG. 7B, the electrodes 140 at the lower left and the upper left of the nanowell 150 are assigned as the electrodes 140A and 140AA with a high voltages, respectively. Furthermore, the electrodes 140 on the upper right and lower right of the nanowell 150 are respectively assigned as the electrodes 140B and 140BB with a low voltage. Therefore, the direction of dipole 205 in the nanowell 150 is from the left to the right. In other words, compared to FIG. 7A, the direction of dipole 205 is rotated 90 degrees clockwise.

FIG. 7C shows a schematic diagram illustrating the electrode 140 assigned at the seventh time t7. In FIG. 7C, the upper left and upper right electrodes 140 of the nanowell 150 are respectively assigned as the electrodes 140A and 140AA with a high voltage. Furthermore, the electrodes 140 at the lower right and the lower left of the nanowell 150 are respectively assigned as the electrodes 140B and 140BB with a low voltage. Therefore, the direction of dipole 205 in nanowell 150 is from above to below. In other words, compared to FIG. 7B, the direction of dipole 205 is rotated 90 degrees clockwise.

FIG. 7D shows a schematic diagram illustrating the electrode 140 assigned at the eighth time t8. In FIG. 7D, the electrodes 140 on the upper right and the lower right of the nano ell 150 are respectively assigned as the electrodes 140A and 140AA with a high voltage. Furthermore, the electrodes 140 at the lower left and the upper left of the nanowell 150 are respectively assigned as the electrodes 140B and 140BB with a low voltage. Therefore, the direction of the dipole 205 in the nanowell 150 is from the right to the left. In other words, compared to FIG. 7C, the direction of dipole 205 is rotated 90 degrees clockwise.

Referring to FIGS. 7A through 7D together, by periodically assigning four electrodes 140 to the corresponding voltages at the fifth time t5, the sixth time t6, the seventh time t7, and the eighth time t8 in sequence, the dipole 205 in the nanowell 150 is rotated. For example, rotate clockwise. It should be noted that a fourth time difference Δt4 from the fifth time t5 to the sixth time t6 is the same as a fifth time difference Δt5 from the sixth time t6 to the seventh time t7, and the fifth time difference Δt5 is the same as a sixth time difference Δt6 from the seventh time t7 to the eighth time t8. Furthermore, when the dipole 205 rotates, the image-sensing cell 104 can detect the change of the object to be tested, and then obtain the dipole moment and the moment of inertia of the object to be tested. Therefore, the image-sensing device 100 can identify the object to be tested more quickly according to the characteristics of the object to be tested.

In some embodiments, the voltage controller (not shown) of the image-sensing device 100 can assign the bias voltage of the electrodes 140 flexibly according to the configuration of the electrode 140 shown in the third bias mode of FIGS. 6A through 6D and the fourth bias mode of FIGS. 7A through 7D, so as to control the direction (clockwise or counterclockwise) and angle (45 degrees, 90 degrees, 135 degrees, etc.) of the rotation of the dipole 205. In addition, in some embodiments, the voltage controller (not shown) of the image-sensing device 100 can divide the nanowell array into multiple regions, and the electrode 140 in each region corresponds to a respective bias mode.

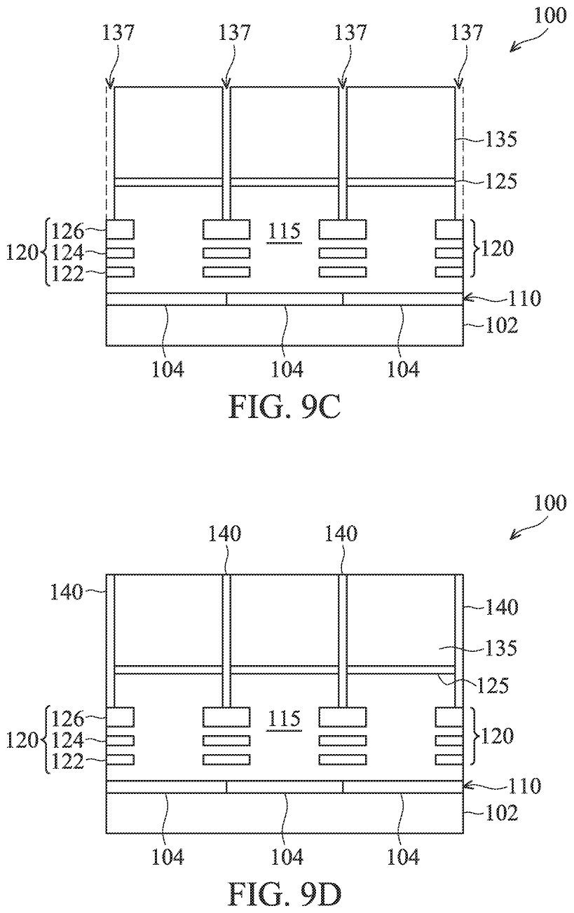

FIG. 8 shows a cross-sectional view of the nanowell 150 and the surrounding electrodes 140 according to some embodiments of the invention. In FIG. 8, the electrode 140 on the left of the nanowell 150a is assigned as the electrode 140B with a low voltage, and the electrode 140 on the right of the nanowell 150a is assigned as the electrode 140A with a high voltage. Therefore, the direction of the dipole 205 in the nanowell 150a is from the right electrode 140A to the left electrode 140B. In addition, the electrode 140 on the left of the nanowell 150b is assigned as the electrode 140A with a high voltage, and the electrode 140 on the right of the nanowell 150b is assigned as the electrode 140B with a low voltage. Therefore, the direction of the dipole 205 in the nanowell 150b is from the electrode 140A on the left to the electrode 140B on the right.

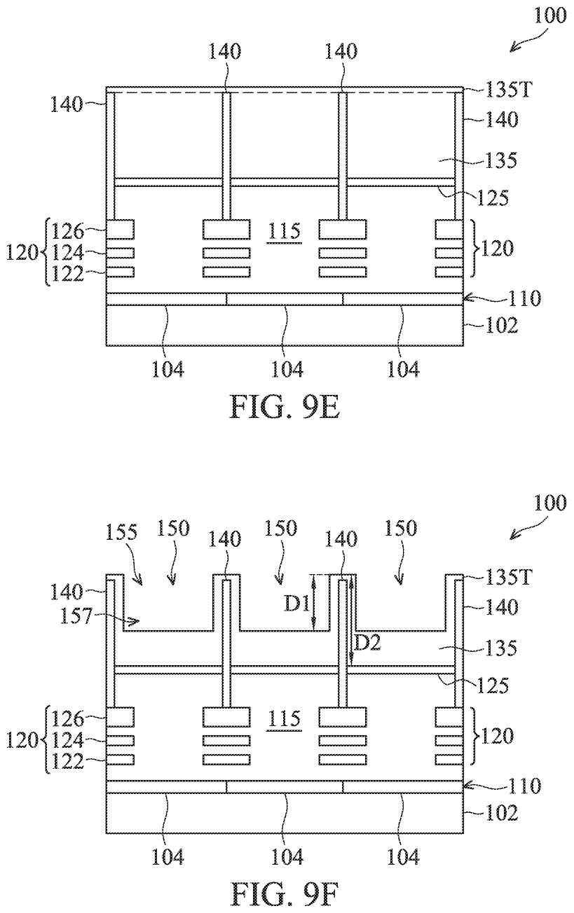

FIGS. 9A through 9F show cross-sectional views illustrating a semiconductor structure forming the image-sensing device 100 according to some embodiments of the invention.

As shown in the cross-sectional view of FIG. 9A. the image sensor array 110 is formed on the substrate 102, and the image sensor array 110 is formed by a plurality of image-sensing cells 104. In some embodiments, some components of the image-sensing cell 104 are formed in the substrate 102. In addition, the dielectric layer 115 is formed on the image sensor array 110, and the interconnect structure 120 is disposed in the dielectric layer 115. As described above, the interconnect structure 120 includes a plurality of conductive layers 122, 124, and 126. Furthermore, the passivation layer 125 is formed on the dielectric layer 115.

As shown in the cross-sectional view of FIG. 9B, the dielectric layer 135 is formed on the passivation layer 125. In some embodiments, the dielectric layer 115 and the dielectric layer 135 are formed of the same dielectric material. In some embodiments, the dielectric layer 115 and the dielectric layer 135 are formed of different dielectric materials. Moreover, the dielectric layer 115 and the dielectric layer 135 are formed by a deposition process.

As shown in the cross-sectional view of FIG. 9C, a mask (not shown) is used to perform an etching process on the dielectric layer 135, the passivation layer 125, and the dielectric layer 115, so as to form the trench 137. Furthermore, the bottom of the trench 137 exposes the upper surface of the conductive layer 126 of the interconnect structure 120.

As shown in the cross-sectional view of FIG. 9D, a conductive material (such as tungsten) is filled into the trench 137 to form the electrode 140. As described above, the electrode 140 is contact with and electrically connected to the conductive layer 126 of the interconnect structure 120. In some embodiments, the electrode 140 may be the via.

As shown in the cross-sectional view of FIG. 9E, a top dielectric layer 135T is formed on the dielectric layer 135 and the electrode 140. In some embodiments, the dielectric layer 135 and the top dielectric layer 135T are formed of the same dielectric material. In addition, the top dielectric layer 135T is formed by a deposition process. By forming the top dielectric layer 135T on the electrode 140, the electrode 140 can be prevented from being electrically connected to other structures (not shown) on the upper layer.

As shown in the cross-sectional view of FIG. 9F, a mask (not shown) is used to perform an etching process on the dielectric layer 135 and the top dielectric layer 135T to form a nanowell 150. As previously described, there is a depth (or thickness) DI between the bottom surface 157 of the nanowell 150 and the opening 155, and the dielectric layer 135 has a depth D2 greater than the depth D1, that is, D2>D1. Therefore, in the image-sensing device 100 of FIG. 9F, the biasable electrodes 140 are formed between the nanowells 150. In some embodiments, each nanowell 150 corresponds to a respective image-sensing cell 104. In some embodiments, each nanowell 150 corresponds to a plurality of image-sensing cells 104.

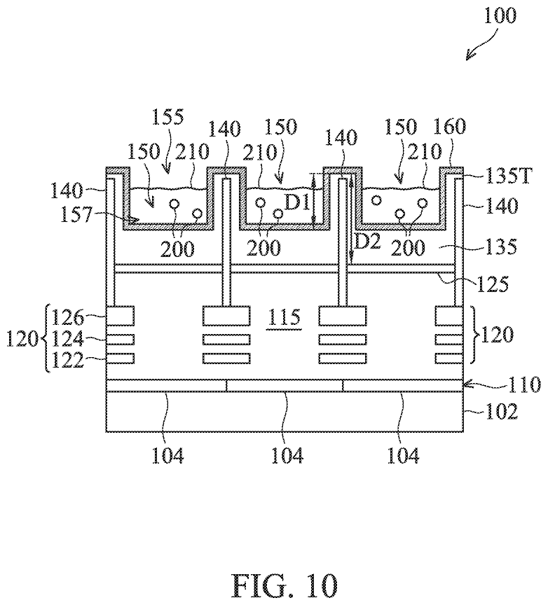

FIG. 10 shows a cross-sectional view of a semiconductor structure forming the image-sensing device 100 according to some embodiments of the invention. In some embodiments, after completing the structure of FIG. 9F, a passivation layer 160 is further formed on the top dielectric layer 135T and the nanowell 150. By forming the passivation layer 160 in the nanowell 150, it is possible to prevent the sample solution (or chemical liquid) 210 from corroding the dielectric layer 135.

According to the embodiments of the invention, by controlling the bias voltage of the electrodes 140, different electric field strengths are formed in the individual nanowells 150, thereby controlling the dipole moment of the object to be tested 200. In addition, the structure of the nanowell 150 and the material of the dielectric layer 135 also affect the electric field strength. Compared with traditional image-sensing devices that cannot apply an electric field to the nanowell or can only apply an electric field to the entire nanowell array, the embodiments of the invention provides an individual electric field for each nanowell by changing the bias voltage of the electrodes 140, to detect whether the light signal intensity and spatial distribution of the light emitted by the object to be tested 200 are stable by the image sensing cell 104, so as to obtain the relaxation time. Next, the image-sensing device 100 obtains the dipole moment and the moment of inertia of the object to be tested 200 according to the relaxation time corresponding to different electric field strengths. Then, according to the ratio of the dipoles moment and the moment of inertia, the image-sensing device 100 can obtain additional information to accelerate the identification of the object to be tested 200.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An image-sensing device, comprising:
a substrate;
a first dielectric layer formed on the substrate, and having a first side and a second side opposite to the first side;
an image sensor array formed between the substrate and the second side of the first dielectric layer, and comprising a plurality of image-sensing cells;
a plurality of nanowells formed in the first dielectric layer, wherein each of the nanowells has an opening on the first side of the -first dielectric layer; and
a plurality of electrodes, wherein each of the electrodes extends from the second side to the first side of the first dielectric layer and is located between two adjacent nanowells.

2. The image-sensing device as claimed in claim 1, further comprising:
a second dielectric layer formed between the first dielectric layer and the image sensor array;
an interconnect structure formed in the second dielectric layer; and
a first passivation layer formed between the first dielectric layer and the second dielectric layer.

3. The image-sensing device as claimed in claim 2, wherein the interconnect structure includes a plurality of conductive layers, and each of the electrodes is disposed on the conductive layer adjacent to the second side of the first dielectric layer and extends to the first side of the first dielectric layer through the first passivation layer.

4. The image-sensing device as claimed in claim 1, wherein when at least one object to be tested is filled into the nanowells, the image-sensing device controls voltages of the electrodes to obtain dipole moment or moment of inertia of the object to be tested, so as to identify the object to be tested.

5. The image-sensing device as claimed in claim 4, wherein the object to be tested comprises a biological molecule, a chemical molecule, or a combination thereof.

6. The image-sensing device as claimed in claim 1, wherein each of the nanowells is surrounded by two of the electrodes with different voltages.

7. The image-sensing device as claimed in claim 1, wherein each of the nanowells is surrounded by a first electrode, a second electrode, a third electrode, and a fourth electrode of the electrodes, wherein the first electrode and the third electrode have an average voltage, and the second electrode has a maximum voltage, and the fourth electrode has a minimum voltage, wherein a direction of dipole of an object to be tested in the nanowell is from the second electrode to the fourth electrode.

8. The image-sensing device as claimed in claim 1, wherein each of the electrodes is disposed between at least four of the nanowells.

9. The image-sensing device as claimed in claim 1, further comprising:
a second passivation layer formed in the nanowells and on the first side of the first dielectric layer.

10. The image-sensing device as claimed in claim 1, wherein a shape of the nanowell is an equilateral polygon or a circle.

11. An image-sensing device, comprising:
a substrate;
an image sensor array formed on the substrate, and comprising a plurality of image-sensing cells;
a first dielectric layer formed on the image sensor array;
a first passivation layer formed on the first dielectric layer;
a second dielectric layer formed on the first passivation layer;
a plurality of nanowells formed in the second dielectric layer, wherein each of the nanowells has an opening on upper surface of the second dielectric layer; and
a plurality of electrodes, wherein each of the electrodes extends from the first dielectric layer through the first passivation layer to the second. dielectric layer and is disposed between two adjacent nanowells.

12. The image-sensing device as claimed in claim 11, further comprising:
an interconnect structure formed in the first dielectric layer,
wherein the interconnection structure comprises a plurality of conductive layers, and each of the electrodes is disposed on the conductive layer adjacent to the second dielectric layer, and extends to the upper surface of the first dielectric layer through the first passivation layer.

13. The image-sensing device as claimed in claim 11, wherein each of the nanowells corresponds to a respective image-sensing cell.

14. The image-sensing device as claimed in claim 11, wherein when at least one object to be tested is filled into the nanowells, the image-sensing device controls voltage of the electrodes to obtain dipole moment or moment of inertia of the object to be tested, so as to identify the object to be tested.

15. The image-sensing device as claimed in claim 14, wherein the object to be tested comprises a biological molecule, a chemical molecule, or a combination thereof.

16. The image-sensing device as claimed in claim 11, wherein each of the nanowells is surrounded by two of the electrodes with different voltages.

17. The image-sensing device as claimed in claim 11, wherein each of the nanowells is surrounded by a first electrode, a second electrode, a third electrode, and a fourth electrode of the electrodes, wherein the first electrode and the third electrode have an average voltage, the second electrode has a maximum voltage, and the fourth electrode has a minimum voltage, wherein a direction of dipole of an object to be tested in the nanowell is from the second electrode to the fourth electrode.

18. The image-sensing device as claimed in claim 11, wherein each of the electrodes is disposed between at least four of the nanowells.

19. The image-sensing device as claimed in claim 11, further comprising:
a second passivation layer formed in the nanowells and on the first side of the first dielectric layer.

20. The image-sensing device as claimed in claim 11, wherein a shape of the nanowell is an equilateral polygon or a circle.
